(19) United States
(12) Plant Patent Application Publication
(10) Pub. No.: US 2005/0257304 P1
(43) Pub. Date: Nov. 17, 2005

(54) Monarda plant named 'Coral Reef'

(50) MONARDA DIDYMA; MONARDA

(76) Inventor: Campbell G. Davidson, Morden (CA)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 10/844,339

(22) Filed: May 13, 2004

(51) Int. Cl. A01H 5/00

(52) U.S. Cl. PLT/263

(57) ABSTRACT

A new and distinct Monarda or Bee-Balm cultivar is provided that displays an upright mounded growth habit and frequently assumes a mature height of approximately three to four feet. Attractive bright coral pink blossoms commonly are formed from June through August. The foliage is dark green, contrasts well with the blossom coloration, and displays a strong minty fragrance. Good resistance to Powdery Mildew is displayed. The plant is well suited for growing as attractive ornamentation in the landscape.

### BOTANICAL COMMERCIAL CLASSIFICATION

Monarda didyma/Monarda Plant

### VARIETAL DENOMINATION

cv. ‘Coral Reef’

### SUMMARY OF THE INVENTION

The new and distinct Monarda (Bee-Balm) cultivar of the present invention was the product of a controlled breeding program carried out at Morden, Manitoba, Canada. The cross that produced the new variety was made in 1990. The female parent was Monarda didyma ‘Mahogany’ (non-patented in the United States), and the male parent was Monarda didyma ‘Marshall's Delight’ (non-patented in the United States). See HortScience, 24 (3) 525 (1989) where the ‘Marshall's Delight’ cultivar is discussed. During 1993 a single plant of the new variety was selected and initially was designated No. 9301 and subsequently Baileef. This plant was further studied in the field evaluations during 1994, 1995, 1996, and 1999. It was found that the new Monarda plant of the present invention exhibits the following combination of characteristics:

(a) forms attractive bright coral pink blossoms,

(b) forms attractive dark green leaves that display a strong minty fragrance,

(c) displays an upright mounded growth habit and commonly assumes a mature height of approximately three to four feet,

(d) grows well in U.S.D.A. Hardiness Zone Nos. 3 to 8,

(e) displays good resistance to Powdery Mildew, and

(f) is well suited for growing as attractive ornamentation in the landscape.

The bright coral pink blossom coloration contrasts nicely with the dark green foliage coloration, is believed to be unique, and causes the plant to stand out in the landscape. The new plant performs well in full sun and no major insect problems have been encountered. The foliage possesses a slight gloss. The mint fragrance of the leaves commonly is very strong.

The new cultivar can be readily distinguished from its ancestors through an inspection of the blossom coloration. More specifically, the ‘Mahogany’ female parent forms bright red blossoms, and the ‘Marshall's Delight’ cultivar forms dissimilar pink blossoms.

The new cultivar of the present invention well meets the needs of the horticultural industry and suitable for growing in the landscape as a specimen or in a mass planting.

The new cultivar has been asexually reproduced by the rooting of cuttings at Morden, Manitoba, Canada, and at St. Paul, Minn., U.S.A. The combination of characteristics exhibited by the new plant has been found to be stable and is reliably transmitted to succeeding generations following such asexual reproduction. Accordingly, the new cultivar reproduces true to type by such asexual propagation.

The new cultivar of the present invention has been named ‘Coral Reef’.

### BRIEF DESCRIPTION OF THE PHOTOGRAPHS

The accompanying photographs show, as nearly true as it is reasonably possible to make the same in color illustrations of this character, typical specimens of the new cultivar. The Monarda plants were reproduced by the rooting of cuttings, were approximately two and three years of age, and were grown outdoors during the 2003 season at St. Paul, Minn., U.S.A.

FIG. 1 illustrates at the center a mature plant of the ‘Coral Reef’ cultivar which displays the customary upright and mounded growth habit.

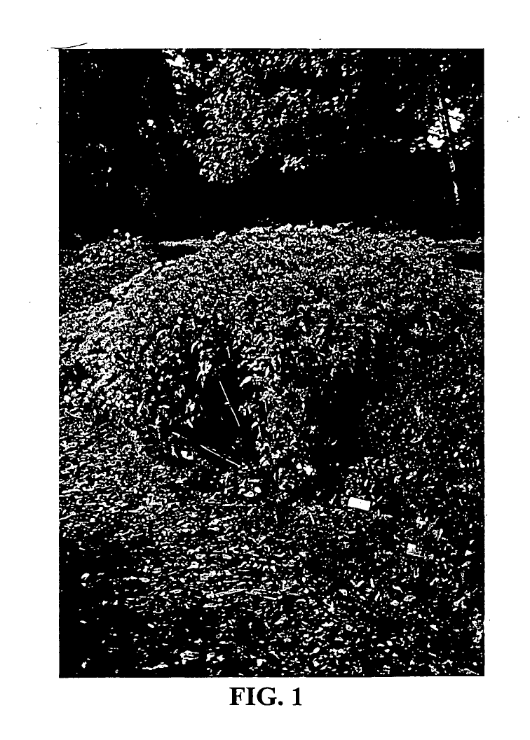

FIG. 2 illustrates a nursery row of plants of the ‘Coral Reef’ cultivar wherein the bright coral pink blossoms are shown in greater detail in the foreground.

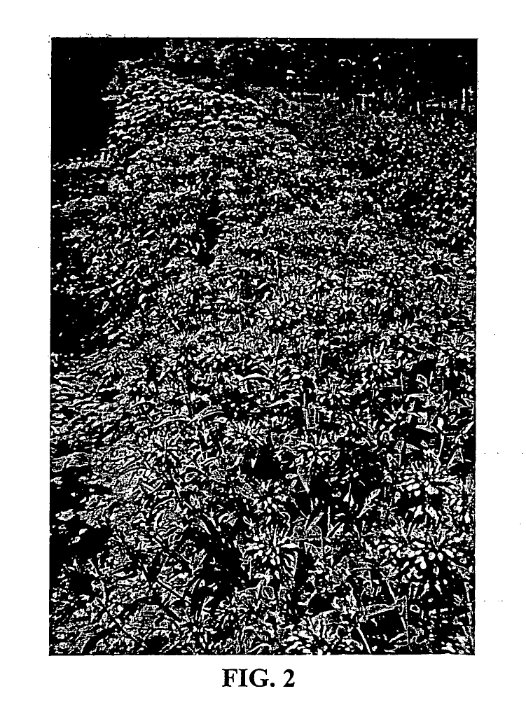

### DETAILED DESCRIPTION

The described plants had been asexually propagated by the rooting of cuttings, were approximately one and two years of age, and were observed during June and August, 2003 while growing outdoors at St. Paul, Minn., U.S.A. The chart used in the identification of color is the R.H.S. Colour Chart of The Royal Horticultural Society, London, England. Reference to common color terms is to be accorded ordinary dictionary significance.
Botanical classification: Monarda didyma cv. ‘Coral Reef’.
Plant:
   Growth habit.—Upright and mounded in configuration.
   Size.—Commonly approximately 90 cm to 1.25 m (approximately 3 to 4 feet) in height on average when mature, and approximately 60 cm to 80 cm (approximately 2 to 2.5 ft) in width on average when mature.
Foliage:
   Configuration.—Ovate to ovate-lanceolate.
   Arrangement.—Opposite.
   Apex.—Acuminate.
   Base.—Rounded to cordate.
   Size.—Leaves commonly measure approximately 7 cm in length on average and approximately 3.25 cm in width on average. The leaf size is similar to that of the ‘Marshall's Delight’ cultivar.
   Margins.—Serrulate.
   General appearance.—Medium density, dark green with a slight gloss. The foliage possesses less glossiness than the ‘Petite Wonder’ cultivar (U.S. Plant Pat. No. 13,149).
   Color.—Young Foliage: Yellow-Green Group 144A on the upper and under surfaces. — Adult Foliage: Commonly between Yellow-Green Group 147A and 147B on the upper surface and between Yellow-Green Group 146B and 147B on the under surface.
   Stems.—Four-sided in structure, and the coloration is Yellow-Green Group 144B and 144C with highlights of Red-Purple Group 59A and 59B on young stems and between Yellow-Green Group 144B and 144C with highlights of Greyed-Orange Group 177A on adult wood.
Inflorescence:
   Natural flowering season.—Commonly heavy blooming from June through August. Such bloom period can be extended by deadheading accompanied by a lesser bloom density.
   Borne.—Flowers are born terminally in single or double whorls sometimes termed verticillasters creating dense heads.
   Configuration.—Composed of substantially tubular florets.
   Size.—A flower diameter of approximately 7.5 cm on average commonly is observed. The petals commonly are approximately 1.75 to 2.5 cm in length and approximately 2 to 4 mm in diameter.
   Color.—When opening begins the upper petal surface is Red Group 54A and the under petal surface is Red-Purple Group 58A, and when blooming the upper petal surface is Red-Purple Group 58B and the under petal surface is Red Group 55B.
   Flower longevity.—Approximately 5 to 7 days when on the plant depending upon environmental conditions.
   Fragrance.—The flowers, stems, and foliage display a strong minty fragrance.
Development:
   Vegetation.—Medium density.
   Culture.—Performs well in full sun.
   Disease resistance.—Good resistance to Powdery Mildew has been observed.
   Insect resistance.—No major insect problems have been encountered.
   Hardiness.—Plants are hardy in U.S.D.A. Hardiness Zone Nos. 3 to 8.
   Propagation.—Plants of the new variety can be readily propagated by the use of rhizome and softwood stem cuttings.
   Usage.—The new variety can be used in traditional formal beds to provide attractive blooms. Large mass plantings are possible. Plants can be used as specimens or as borders defining sections of beds or boundaries. The dark green foliage contrasts well with the bright coral pink flowers.

1. A new and distinct Moranda plant having the following combination of characteristics:
(a) forms attractive bright coral pink blossoms,
(b) forms attractive dark green leaves that display a strong minty fragrance,
(c) displays an upright mounded growth habit and commonly assumes a mature height of approximately three to four feet,
(d) grows well in U.S.D.A. Hardiness Zone Nos. 3 to 8,
(e) displays good resistance to Powdery Mildew, and
(f) is well suited for growing as attractive ornamentation in the landscape;
substantially as illustrated and described.
